(19) United States
(12) Patent Application Publication
(10) Pub. No.: US 2014/0187777 A1
(43) Pub. Date: Jul. 3, 2014

(54) MATERIALS AND METHODS FOR THE BASE-ASSISTED SYNTHESIS OF SUBSTITUTED HETEROAROMATIC COMPOUNDS

(71) Applicant: Dow Agrosciences LLC, (US)

(72) Inventors: Ronald Leng, Midland, MI (US); Gary Roth, Midland, MI (US); Michael Gullo, Midland, MI (US); Stacey Powers, Pinconning, MI (US)

(73) Assignee: Dow Agrosciences LLC, Indianapolis, IN (US)

(21) Appl. No.: 13/848,956

(22) Filed: Mar. 22, 2013

Related U.S. Application Data
(60) Provisional application No. 61/615,616, filed on Mar. 26, 2012.

(51) Int. Cl. C07D 239/47

(52) U.S. Cl. CPC C07D 239/47
USPC 544/317

(57) ABSTRACT

Disclosed herein are some methods for synthesizing the base-assisted coupling of aromatic alcohols with substituted aromatic compounds including some substituted heteroaromatic compounds such as pyrimidines or pyridines to form esters. The reactions may include inexpensive bases such as potassium hydroxide and take place in a solvent mixture comprised primarily of hindered tertiary alcohols that are at least partially immiscible with water. In some embodiments the tertiary alcohol solvents forms azeotropes with water which stabilize the formation of an alkoxide intermediate when the alkoxide formation step is heated to the boiling point of the azeotrope and water is withdrawn from the reaction mixture.

### PRIORITY CLAIM

This application claims the benefit of U.S. provisional patent application No. 61/615,616 filed on Mar. 26, 2012, which is incorporated herein by reference in its entirety.

### FIELD OF THE INVENTION

The invention relates generally to materials and methods for promoting base-assisted coupling of aromatic or heteroaromatic compounds such as pyrimidines with alcohols via the formation of an alkoxide formed in the presence of a solvent that forms an azeotrope with water.

### BACKGROUND

Coupling reactions between halogenated aromatic compounds and alcohols may proceed through the formation of an alkoxide. Stability of alkoxides formed between alcohols and simple bases such as potassium and sodium hydroxide may be a problem because water that forms as part of the dehydration reaction can react with the alkoxide before it can couple with the aromatic compound. Hydroxide can then react with the halogenated aromatic compound resulting in phenols or heterocyclic hydroxy compounds. The utility of base-promoted aromatic coupling between halogentated aromatic compounds and alcohols can be increased if the half-life of the alkoxide intermediate can be extended. Some aspects of the invention address these needs by providing combinations of reagents and/or process conditions that promote these types of coupling reactions.

### SUMMARY

Some aspects of the invention include methods for conducting reactions between aromatic compounds such as 2-chloro-5-fluoro-4-aminopyrimidine (CFAP) and substituted alcohols such as 4-methyl benzyl alcohol (MBA) wherein the reaction is carried out in a solvent that includes tertiary amyl alcohol (t-AA) and an inorganic base such as potassium hydroxide (KOH).

Some embodiments of the invention include methods for conducting base-assisted reactions, comprising the steps of: forming an alkoxide by: mixing at least one alcohol with at least one metal hydroxide in the presence of a tertiary alcohol, wherein the tertiary alcohol is at least partially immiscible with water; and contacting the alkoxide with an aromatic compound. In some embodiments the aromatic compound is substituted aromatic with an compound, and halogen substituted aromatic compound undergoes a base-assisted coupling with said alkoxide to form an ether. 2. The method according to claim 1, wherein the substituted aromatic compound is a heteroaromatic compound. 3. The method according to claim 2, wherein the heteroaromatic compound is selected from the group consisting of: pyrmidines and pyridines. 4. The method according to claim 1, wherein tertiary alcohol at least partially immiscible with water also forms an azeotrope with water. 5. The method according to claim 4, wherein the tertiary alcohol is tert-amyl alcohol. 6. The method according to claim 1, wherein the aromatic alcohol is 4-methyl benzyl alcohol. 7. The method according to claim 1, wherein the metal hydroxide is selected from the group consisting of: lithium hydroxide, sodium hydroxide, potassium hydroxide, and cesium hydroxide. 8. The method according to claim 1, wherein the halogen substituted aromatic compounds is 2-chloro-5-fluoro-4-aminopyrimidine. 9. The method according to claim 8, wherein the alcohol is 4-methyl benzyl alcohol. 10. The method according to clam 2, further including the steps of:
drying the solution that includes the alkoxide by heating said solution to the boiling point of the azeotropic mixture. 11. The method according to claim 1, further including the step of:
washing the mixture with an aqueous solvent. 12. The method according to claim 11, wherein the solvent is water. 13. The method according to claim 11, further including the step of:
recovering the product of the base-assisted coupling of the aromatic compound with the aromatic alcohol by processing the organic phase of the wash. 14. The method according to claim 10, further comprising the step of:
concentrating the product by evaporating at least a portion of the solvent and cooling the product. 15. The method according to claim 10, including the steps of:
washing the product with a hindered alcohol solvent;
recovering the hindered alcohol solvent;
drying the recovered solvent; and
re-using the dried recovered solvent in the process.

### BRIEF DESCRIPTION OF THE FIGURES

FIG. 1 Block diagram illustrating steps in the reaction of an aromatic compound such as 2-chloro-5-fluoro-4-aminopyrimidine (CFAP) with an alcohol such as 4-methyl benzyl alcohol (MBA) in a solvent comprising a hindered tertiary alcohol such as t-amyl alcohol.

### DESCRIPTION

For the purposes of promoting an understanding of the principles of the novel technology, reference will now be made to the preferred embodiments thereof, and specific language will be used to describe the same. It will nevertheless be understood that no limitation of the scope of the novel technology is thereby intended, such alterations, modifications, and further applications of the principles of the novel technology being contemplated as would normally occur to one skilled in the art to which the novel technology relates are within the scope of this disclosure and the claims.

As used herein, unless explicitly stated otherwise or clearly implied otherwise the term ‘about’ refers to a range of values plus or minus 10 percent, e.g. about 1.0 encompasses values from 0.9 to 1.1.

Base-induced reactions between substituted aromatic compounds and alcohols may take place via the formation of an alkoxide. Alkoxides are reactive towards aromatic compounds substituted with for example halogens such as chlorine, fluorine and the like. The formation of an alkoxide between a simple metal hydroxide, such as KOH and a stable alcohol results in formation of the alkoxide and water. Unfortunately, many alkoxides are not very stable in the presence of water. In order to increase the utility of certain base-assisted coupling reactions which proceed through the formation of an alkoxide, some embodiments of the invention include forming and/or stabilizing the alkoxide by forming it or at least extending its half-life by holding it in a solution that includes at least one compound that forms an azeotrope with water. In some embodiments the azeotropic mixture is removed from the reaction by heating. Because removal of the water-based azeotrope necessarily reduces the level of water in contact with the reactive alkoxide, the combination of forming the azeotrope and heating the mixture to the boiling point of the azeotrope has the effect of increasing the efficiency of reactions that proceed through the formation of an alkoxide intermediate.

Exemplary reactions that can benefit form the materials and methods disclosed herein include reactions that form substituted ethers via the reaction of an alkoxide with a substituted alkyl halide (RX+R′O⁻ to ROR′ the Williamson ether synthesis). Still other reactions that can benefit from the inventive methods include reactions that requires a water free primary or secondary alkoxide the conjugate acid of which has a boiling point above that of the tertiary alcohol.

The invention can be practiced using, for example, halogenated aromatics, halo-pyridines, halopyrimidines and other halogenated heteroaromatics and the like.
Simple bases that can be used in the inventive methods include sodium hydroxide (NaOH), lithium hydroxide (LiOH) and potassium hydroxide (KOH).

Azeotropes that are especially useful for the practice of the invention include positive, heterogeneous azeotropes especially those that have boiling points below the boiling point of water, and can be at least partially phase separated in the resultant condensed distillate. Still another trait of many of these useful azeotropes is that the compounds combined with water to form the azeotrope are stable under the conditions of the reactions and do not themselves readily react with the principle reactants or products under the conditions under which the reactions conducted.

Hindered alcohols that can be used to practice the invention include, but are not limited to alcohols such as t-butanol.

Some embodiments of the invention include the step of adding tertiary alcohols that are at least partially immiscible with water to the reaction mixture. In some preferred embodiments these compounds may form a positive azeotrope with water that is present or formed under reaction conditions.

In some embodiments, a stable alcohol that is at least partially immiscible with water promotes a base-assisted coupling reaction even if it does not form an azeotrope. The presence of the proper type of alcohol in the reaction mixture may serve to reduce the effective concentration of water in the reaction by essentially diluting it out, thereby reducing the effective concentration of water in the reaction. Reducing the level of water in the reaction mixture has the effect of decreasing the rate at which water present in the reaction (or formed during the reaction) interferes with the reaction.

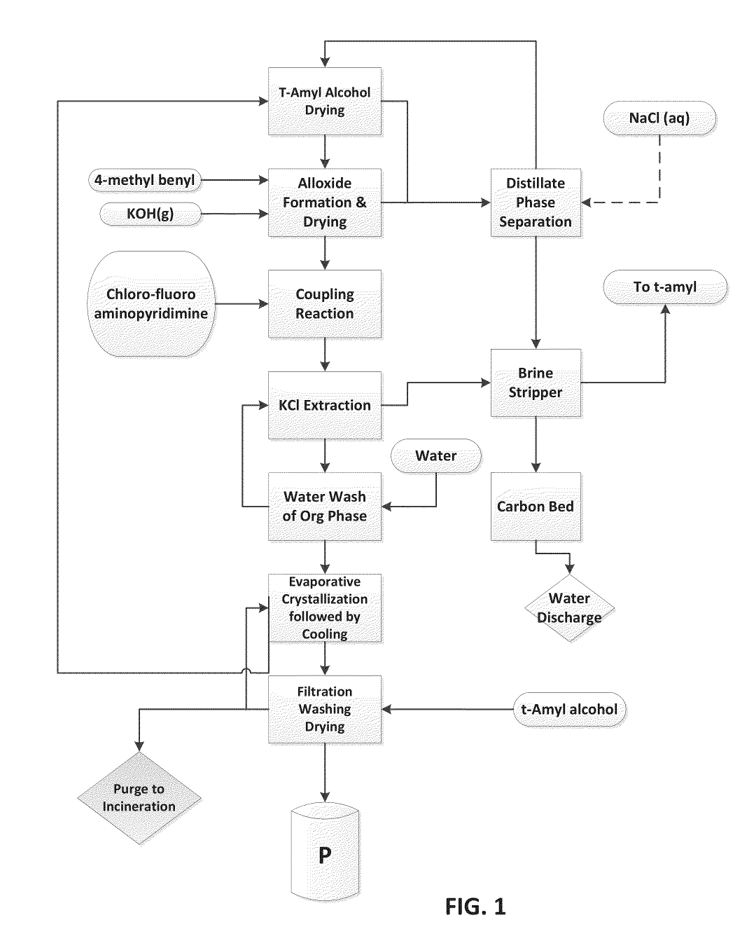

Referring now to FIG. 1, an exemplary synthesis of compounds such as Compound:

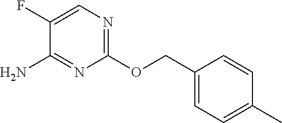 [SMILES: Cc1ccc(COc2ncc(F)c(N)n2)cc1]

These types of compounds can be formed by base-assisted coupling of halogen substituted aromatic compounds with alcohols. Aromatic compounds that are substituted with at least one halogen include heteroaromatic compounds such as pyrimidines and pyridines. Alcohols that can couple with halogen substituted compounds include, but are not limited to, aromatic alcohols and substituted secondary and primary alcohols. One exemplary reaction is that which occurs between 2-chloro-5-fluoro-4-aminopyridimine (CFAP) and 4-methyl benzyl alcohol (MBA) in the presence of a simple base such as potassium hydroxide.

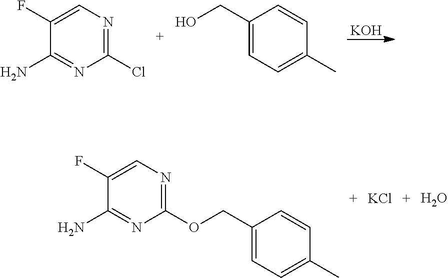

Briefly, dried, recycle t-AA, MBA, and KOH (flake) are loaded into the drying vessel, which is connected to a batch rectification column. The mixture is heated with stirring to boiling. The reflux rate is adjusted to rectify the distillate to the azeotropic composition of about 28% water. As water is removed, it is harder to maintain the azeotropic composition; accordingly, there is an optimization point on reflux rate and time. The endpoint is determined by distillation temperatures.

The distillation mixture is cooled and CFAP is added at the appropriate stoichiometric ratio (nominally 1.2-1.5 eq MBA/eq CFAP) The reaction is held at about 80-90° C. for about 8-12 hours or until the CFAP conversion is at target as confirmed by liquid chromatography (LC) analysis. Following completion of the reaction water is added to extract potassium chloride (KCl). The amount of water added is adjusted to target a ˜20% KCl solution. The contents are mixed for a period of time then allowed to settle. The brine phase is decanted to the post treatment area.

A second portion of water is added to wash the organic phase to further reduce salts and water-soluble impurities. The contents are mixed and settled and again the aqueous phase is withdrawn as the bottom phase. This “weak brine” can be reserved and used as the primary wash for the next batch, or it may be discarded. Addition of a small amount of salt may be beneficial for improving phase separation. The organic phase is heated to evaporate a portion of the solvent. Residual water will azeotrope off which will lower the solubility of the Compound 1. Some vacuum may be applied to control the evaporation at a lower temperature.

Following the concentration step, the solution is cooled slowly to crystallize Compound 1. The batch is cooled to <10° C. to reduce the solubility of Compound 1 to a lower level. The cooled Compound 1 slurry is fed into a centrifuge or suitable filter to separate the crystals from the liquid. The filter cake is washed with a portion of chilled t-AA. The filter cake is placed in a dryer to remove residual solvent affording dry Compound 1. The filtrate liquors are combined and a portion discarded to manage the build up of impurities. The balance of the liquors may be recycled to the next crystallization step or directly to the reaction step to increase the yield of Compound 1. Alternatively, the filtrates can be evaporated and cooled to harvest a second crop of product.

Wet t-AA distillates are collected and are azeotropically redistilled to remove water. An option method is to add some salt (e.g. sodium chloride (NaCl) or potassium chloride (KCl)) in order to reduce the water content in the t-AA by salting effect. The dried solvent is then available to be recycled to the next reaction sequence if desired.

### Experimental

### Materials and Methods

### Example 1

A 500-mL round bottomed flask is equipped with overhead stirring and a 22 tray×1″ Oldershaw column. To the flask is added tert-amyl alcohol (245 grams (g), 2.78 moles (mol), 7.9 equivalents (eq.) based on CFAP), 4-methylbenzyl alcohol (63 g., 0.52 mol, 1.5 eq. based on CFAP), and flake KOH (90%; 31.1 g. 0.5 mol, 1.5 eq. based on CFAP). The mixture is stirred and heated to boiling. The system is held at total reflux for about 30 minutes (min.) to equilibrate the Oldershaw column, after which distillation is begun with a reflux ratio of 20:1. After about 8.5 hours (h.), Karl Fisher titration of the bottoms indicates about 0.82% water. Heating is stopped. Once the mixture reaches room temperature, CFAP is added (49.6 g., 0.34 mol). The mixture is heated to and held at about 80-90° C. and sampled periodically for high-performance liquid chromatography (HPLC) analysis. After 10.75 h. at reaction temperature, CFAP is <0.5% by area and heating is stopped. Once the mixture reaches 65° C., water (124.6 g.) is added, which cools the mixture to about 48° C. The mixture is stirred while warming it back to about 65° C. The warm mixture is transferred to a separatory funnel. The brine layer is cut, and the organic layer (that includes a small rag layer) is returned to the distillation flask. To the stirring organic layer is added an additional water wash (about 124.6 g.), and the mixture is warmed to 65° C., transferred to a separatory funnel, and left overnight for phase separation. During the cooling period, Compound 1 crystallizes in the organic phase. The aqueous phase is cut, and the organic phase containing the crystals is heated to dissolve the solids and enable easier removal from the separatory funnel. The organic phase (about 225 g.) is transferred to a flask and concentrated by rotary evaporation to about 129.1 g. As the product begins to precipitate during the concentration, vacuum is discontinued and the rotary evaporator bath is heated to about 75° C. to re-dissolve the solids. The flask is removed from the rotary evaporator, overhead stirring is added, and the flask is wrapped with insulation to allow for a more gradual cool down period. Once the mixture had reached room temperature, the solids are isolated by filtration. The wet cake (about 63.1 g.) is washed with cold tert-amyl alcohol (58.9 g), and the washed cake is dried in the fume hood. HPLC internal standard analysis is carried out on the dried solid (50.8 g., 98.5% Compound 1, 0.21 mol), the mother liquor (48.7 g., 23.9% Compound 1, 0.05 mol.), and the cake wash (62.3 g., 11.2% Compound 1, 0.03 mol).

### Example 2

A second reaction is carried out under conditions similar to those disclosed in Example 1. Briefly, the starting materials in this example are 2.78 mol tert-myl alcohol, 0.51 mol. 4-methylbenzyl alcohol, 0.45 mol. KOH. Upon reaction completion, the mixture is treated with water in the same manner as the previously described experiment. The organic phase (257.1 g.) is transferred to a flask and concentrated to 154.1 g. by rotary evaporation (in order to keep product in solution the bath temperature set to about 70° C.). The flask is removed from the rotary evaporator, overhead stirring is added, and the flask is wrapped with insulation to allow for allow for a more gradual cool down period. Once the mixture reaches room temperature, the mixture is cooled with an ice bath to about 2° C. The solids are isolated by filtration. The filtration rate is noticeably slower than observed when filtering the slurry at room temperature. The wet cake is washed with cold tert-myl alcohol (about 78.6 g). A slower filtration rate is also observed in this step. The washed cake is dried in the fume hood. HPLC internal standard analysis is carried out on the dry solids (about 72.2 g., 93.6% Compound 1, 0.29 mol.), the mother liquor (23.3 g., 15.1% Compound 1, 0.02 mol.), and the cake wash (77.6 g., 9.0% Compound 1, 0.03 mol.).

While the novel technology has been illustrated and described in detail in the figures and foregoing description, the same is to be considered as illustrative and not restrictive in character, it being understood that only the preferred embodiments have been shown and described and that all changes and modifications that come within the spirit of the novel technology are desired to be protected. As well, while the novel technology was illustrated using specific examples, theoretical arguments, accounts, and illustrations, these illustrations and the accompanying discussion should by no means be interpreted as limiting the technology. All patents, patent applications, and references to texts, scientific treatises, publications, and the like referenced in this application are incorporated herein by reference in their entirety.

We claim:

1. A method for conducting base-assisted reactions, comprising the steps of:
forming an alkoxide by:
mixing at least one alcohol with at least one metal hydroxide in the presence of a tertiary alcohol, wherein said tertiary alcohol is at least partially immiscible with water;
contacting said alkoxide with an aromatic compound, wherein a halogen substituted aromatic compound, and wherein the halogen substituted aromatic compound undergoes a base-assisted coupling with said alkoxide to form an ether.

2. The method according to claim 1, wherein the substituted aromatic compound is a heteroaromatic compound.

3. The method according to claim 2, wherein the heteroaromatic compound is selected from the group consisting of: pyrmidines and pyridines.

4. The method according to claim 1, wherein tertiary alcohol at least partially immiscible with water also forms an azeotrope with water.

5. The method according to claim 4, wherein the tertiary alcohol is tert-amyl alcohol.

6. The method according to claim 1, wherein the aromatic alcohol is 4-methyl benzyl alcohol.

7. The method according to claim 1, wherein the metal hydroxide is selected from the group consisting of: lithium hydroxide, sodium hydroxide, potassium hydroxide, and cesium hydroxide.

8. The method according to claim 1, wherein the halogen substituted aromatic compounds is 2-chloro-5-fluoro-4-aminopyrimidine.

9. The method according to claim 8, wherein the alcohol is 4-methyl benzyl alcohol.

10. The method according to clam 2, further including the steps of:
drying the solution that includes the alkoxide by heating said solution to the boiling point of the azeotropic mixture.

11. The method according to claim 1, further including the step of:
washing the mixture with an aqueous solvent.

12. The method according to claim 11, wherein the solvent is water.

13. The method according to claim 11, further including the step of:
recovering the product of the base-assisted coupling of the aromatic compound with the aromatic alcohol by processing the organic phase of the wash.

14. The method according to claim 10, further comprising the step of:
concentrating the product by evaporating at least a portion of the solvent and cooling the product.

15. The method according to claim 10, including the steps of:
washing the product with a hindered alcohol solvent;
recovering the hindered alcohol solvent;
drying the recovered solvent; and
re-using the dried recovered solvent in the process.
